(19) United States
(12) Patent Application Publication
(10) Pub. No.: US 2023/0183794 A1
(43) Pub. Date: Jun. 15, 2023

(54) POLYNUCLEOTIDE ARRAYS

(71) Applicant: Oxford University Innvovation Limited Buxton Court, Oxford (GB)

(72) Inventors: Adam CRIBBS, Oxford (GB); Martin PHILPOTT, Oxford (GB); Udo OPPERMANN, Oxford (GB); Tom BROWN, JR., Oxford (GB); Tom BROWN, Oxford (GB); Jonathan Francis WATSON, Oxford (GB)

(21) Appl. No.: 17/924,425

(22) PCT Filed: May 13, 2021
(86) PCT No.: PCT/GB2021/051151

§ 371 Date: Nov. 10, 2022

(30) Foreign Application Priority Data
May 13, 2020 (GB) 2007059.5

(51) Int. Cl. C12Q 1/686

(52) U.S. Cl. CPC C12Q 1/686

(57) ABSTRACT

The invention relates to micro-particles in which polynucleotides are joined to a bead at the 3′ end and include a linker that can be cleaved to separate the polynucleotides from the bead and provide free 3′ hydroxyl groups. Also provided are arrays of polynucleotides, pluralities of micro-particles, fluidic compartments comprising micro-particles, methods of synthesising the arrays and methods of generating libraries using the array.

### FIELD OF THE INVENTION

The disclosure relates to arrays of polynucleotides and micro-particles comprising an array of polynucleotides. The arrays and micro-particles find use in, for example, multiplex methods of generating libraries from biological samples, where analytes may be captured at one or both ends of the polynucleotides and amplified to provide libraries for sequencing or other downstream processes.

### BACKGROUND TO THE INVENTION

Understanding cellular physiology and pathobiology requires analysis of the relationship between genotype, chromatin organisation and phenotype. In the multiomics era, many methods exist to investigate biological processes across the genome, transcriptome, epigenome, proteome and metabolome. Until recently, this was only possible for populations of cells or complex tissues, creating an averaging effect that may obscure direct correlations between multiple layers of data. Single-cell sequencing methods have removed this averaging effect, but computational integration after profiling distinct modalities separately may still not completely reflect underlying biology. Multiplexed assays resolving multiple modalities in the same cell are required to overcome these shortcomings and have the potential to deliver unprecedented understanding of biology and disease.

Chen et al. (Nat Biotechnol 37, 1452-1457) have developed a oligonucleotide splint method for multiplexing single cell transcriptomics and chromatin accessibility analysis in single cells, referred to as SNARE-seq [26]. Pooled extracted nuclei are treated with Tn5 transposase prior to encapsulation on a Drop-seq platform. PolyT barcoding beads capture both mRNA directly and transposed DNA via a splint oligo that binds to the polyT oligo at one end and the 5′ overhang of transposed DNA at the other end. After droplets are broken, on-bead reverse-transcription with a template switch oligo and covalent ligation of tagmented DNA to the bead are performed in a single step, followed by simultaneous amplification of both cDNA and transposed DNA. Amplified material can then be split without loss of information. Since amplified cDNA and transposed DNA already contain cellular barcodes, library preparation can proceed independently using standard bulk methods. However, this method has some disadvantages because the splint oligonucleotide efficiency is influenced by temperature and the splint length, which are not always optionally controllable in this assay and both RNA and DNA analytes are competing for the same capture sites. Furthermore, because multiple hybridisation and T7 ligation events need to occur before DNA is fully captured, this can impact on its overall efficiency.

Most single-cell genomics assays have been adapted from techniques developed for analysing bulk-cell populations. Nonetheless, most single-cell sequencing based assays require a minimum level of input material that exceeds that of a single cell and accordingly, amplification strategies and development of instruments that physically capture and isolate individual cells provided the first major advancements. Single cell sequencing methods can be well-based (where a cell is transferred into an individual well of a multi-well plate, which acts as a discrete reaction vessel for subsequent steps), microfluidics i.e. lab-on-a-chip-based (where single cells are held at discrete capture sites on a microfluids chip and some steps of library preparation occur in an automated fashion) or droplet-based (where large numbers on cells are individually captured in droplets within an oil emulsion, which then act as enclosed reaction vessels). Well-based and lab-on-chip-based approaches largely remain limited to interrogating hundreds to the low thousands of cells, but may deliver richer information, including coverage of whole transcripts, detection of lower abundance analytes or measurement of analytes not currently amenable to higher throughput approaches. On the other hand, droplet-based assays are capable of reporting on many thousands of cells, opening up applications not practical with lower cell numbers. However, the use of barcoded oligo beads in these assays bring their own limitations, such as incomplete analyte capture or restriction to end-sequencing of mRNA transcripts.

### SUMMARY OF THE INVENTION

The present inventors have developed means and methods that address some of the limitations of existing barcoded oligo beads used in present methods. Such beads generally comprise a micro-bead and an array of oligonucleotides that are joined to the micro-bead at one end, whilst the opposite, free end is used to capture analyte. Processing steps and reactions are generally carried out with the capture oligonucleotides bound to the bead. When the beads are for capturing RNA, the oligonucleotides are generated on the beads in a 5′ to 3′ direction to provide a free 3′ end hydroxyl group to initiate reverse transcription. However, manufacturing oligonucleotides in a 5′ to 3′ direction is more difficult, more prone to errors, and less flexible than manufacturing oligos in a 3′ to 5′.

The present inventors have developed micro-particles in which the polynucleotides are joined to the bead at the 3′ end and include a linker that can be cleaved to separate the polynucleotides from the bead and provide free 3′ hydroxyl groups. This provides at least three advantages: 1) simplified processing steps of capture polynucleotides after release from beads; un-bound oligonucleotides may also have more opportunity to contact and bind to analytes; 2) analytes can be captured at either end of the polynucleotides after release from bead; this allows for multiplexed library generation from multiple groups of analytes in the same sample, and may increase the efficiency of analyte capture; and 3) RNA capture can be achieved using oligonucleotides that are manufactured on beads in a 3′ to 5′ direction. The oligonucleotides of the invention also include all of the other elements needed for efficient library generation from analytes captured at the free 3′ end and optionally the 5′ end.

Accordingly, in a first aspect, the invention provides a micro-particle comprising a micro-bead and an array of polynucleotides, wherein each polynucleotide is attached to the micro-bead at the 3′ end, and wherein each polynucleotide comprises, in a 3′ to 5′ direction: (a) a linker that is cleavable to provide a free 3′ hydroxyl group on the polynucleotide after cleavage; (b) a 3′ end analyte capture region; (c) a barcode sequence (BC), wherein the BC of each polynucleotide is the same; and a unique molecular identifier sequence (UMI), wherein the UMI of each polynucleotide is different; and wherein the UMI is 5′ or 3′ to the BC; and (d) optionally a PCR handle sequence.

In some cases the micro-particles comprise, in a 3′ to 5′ direction: (a) a linker that is cleavable to provide a free 3′ hydroxyl group on the polynucleotide after cleavage; (b) a 3′ end analyte capture region; (c) optionally a first polymerase chain reaction (PCR) handle sequence; (d) a barcode sequence (BC), wherein the BC of each polynucleotide is the same; and a unique molecular identifier sequence (UMI), wherein the UMI of each polynucleotide is different; and wherein the UMI is 5′ or 3′ to the BC; (e) optionally a second PCR handle sequence; and (f) a 5′ end analyte capture region. After cleavage from the bead, the polynucleotides can bind analyte at the 3′ end and/or the 5′ end and libraries comprising the BC and UMI sequences can be generated from analytes captured at either or both ends.

In some cases the linker comprises a photocleavable linker. In other cases the linker comprises a site for cleavage by one or more enzymes to provide the free 3′ hydroxyl group.

In a further aspect, the invention provides an array of polynucleotides comprising, from 3′ to 5′: (a) a 3′ hydroxyl group; (b) a 3′ end analyte capture region; (c) optionally a first polymerase chain reaction (PCR) handle sequence; (d) a barcode sequence (BC), wherein the barcode sequence of each of the polynucleotides is the same; and a unique molecular identifier sequence (UMI), wherein the UMI of each polynucleotide is different; and wherein the UMI is 5′ or 3′ to the BC; (e) optionally a second PCR handle sequence; and (f) optionally a 5′ end analyte capture region. In some cases, the array of polynucleotides is a group of polynucleotides that have been cleaved from a micro-particle as described above, for example after isolation of a single micro-particle in a fluidic compartment where the polynucleotides contact sample comprising analytes. In other cases the array of micro-particles may be bound to or released from a different type of micro-particle, such as a hydrobead.

In a further aspect, the invention provides a plurality of micro-particles as described above, or a plurality of micro-particles each comprising a micro-bead bound to an array of polynucleotides as described above, wherein the array of each micro-particle has a different barcode sequence from the array of essentially each other micro-particle.

In a further aspect, the invention provides a fluidic compartment, optionally a microfluidic compartment, comprising a single micro-particle or a single array of polynucleotides according to any one of claims 1 to 15 and optionally a single cell, a single cell nucleus, a single cell lysate or a single cell nucleus lysate.

In a further aspect, the invention provides a method of synthesising an array of polynucleotides on the surface of a micro-bead, wherein the polynucleotides are synthesized in a 3′ to 5′ direction from the bead to the polynucleotide free ends and wherein each polynucleotide comprises, from 3′ to 5′: (a) a linker that is cleavable to provide a free 3′ hydroxyl group on the polynucleotide after cleavage from the micro-particle; (b) a 3′ end analyte capture region; (c) optionally a first polymerase chain reaction (PCR) handle sequence; (d) a barcode sequence (BC), wherein the barcode sequence of each of the polynucleotides is the same; and a unique molecular identifier sequence (UMI), wherein the UMI of each polynucleotide is different; and wherein the UMI is 5′ or 3′ to the BC; (e) optionally a second PCR handle sequence; and (f) optionally a 5′ end analyte capture region.

In a further aspect, the invention provides a method for generating one or more libraries from one or more groups of analytes from the same sample, wherein the method comprises (i) contacting the sample with an array of polynucleotides as described above; (ii) allowing analytes to bind to the 3′ end and/or 5′ end analyte capture regions of the polynucleotides; and (iii) generating one or more libraries from the analytes bound to the 3′ end and/or 5′ end analyte capture regions, optionally wherein the method comprises generating one or more first libraries from the analytes bound to the 3′ end analyte capture regions, and generating one or more further/second libraries from the analytes bound to the 5′ end analyte capture regions.

In some cases the 3′ end capture regions bind to RNA in the sample and the method comprises (iv) reverse transcription using the bound RNA as template to provide an RNA/cDNA hybrid; (v) template switch to extend the end of the RNA/cDNA hybrid to include a template switch PCR handle sequence; and (vi) PCR amplification using primers that hybridize to (A) the template switch PCR handle sequence and (B) the PCR handle sequence 5′ to the BC and UMI sequence.

In some cases the 5′ end capture region binds to DNA in the sample, and the method comprises PCR amplification using a pair of PCR primers that hybridize to (A) a PCR handle on the DNA bound to the 5′ end capture region, and (B) the complement of the PCT template handle sequence 3′ to the UMI and BC. In some cases the 3′ end capture region binds to DNA in the sample, and the method comprises PCR amplification using pair of PCR primers comprising (A) a PCR handle on the DNA bound to the 3′ end capture region, and (B) the complement of the PCT template handle sequence 5′ to the UMI and BC.

In some cases the one or more groups of analytes are from a single cell, a single cell nucleus or other cellular membrane compartment such as a vesicle. The method may comprise (i) isolating the single cell, single cell nucleus or vesicle with a single micro-particle as described above in a fluidic compartment; (ii) lysing the cell or cell nucleus; and (iii) cleaving the linker to provide the array of polynucleotides.

In other cases the analytes may be from a sample comprising a plurality of cells or cell nuclei. The method may comprise: (i) isolating a single cell or single cell nuclei of the sample and a single micro-particle as described above in each of a plurality of separate fluidic compartments, wherein the polynucleotide array of essentially each micro-particle has a different barcode sequence; (ii) lysing the isolated cells and/or cell nuclei; and (iii) cleaving the linker of the polynucleotides to provide an array of polynucleotides with free 3′ hydroxyl groups in each fluidic compartment.

The disclosure will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise.

Thus, for example, reference to “a polynucleotide” includes two or more such polynucleotides.

Section headings are used herein for convenience only and are not to be construed as limiting in any way.

### DESCRIPTION OF THE FIGURES

FIG. 1—A typical droplet based sequencing bead design. The oligonucleotide synthesis takes place in the 5′ to 3′ direction and includes a barcode that is specific for each bead, a polyA capture site and a Unique Molecular Identifier (UMI) that is specific for each captured transcript.

FIG. 2—A dual RNA DNA capture bead of the invention, synthesized in the 3′ to 5′ direction. The oligonucleotide contains a barcode that is specific for each bead and a Unique Molecular Identifier (UMI), which are flanked by PCR handles. There is an oligonucleotide sequence on the 3′ end, which is typically a poly T sequence to capture polyA RNA, and an oligonucleotide sequence at the 5′ end, which is typically a DNA capture sequence (e.g. Transposed DNA). The oligonucleotide shown also contains a photocleavable linker at the 3′ end and can also include a hairpin sequence that contains a Uracil base that acts as a cleavage site for APE-1 and UDG enzymes.

FIG. 3—Overview of protocol for dual RNA DNA sequencing library preparation. 1. The oligonucleotide is released from the bead using either a photocleavable linker or a combination of both photocleavable linker and UDG/APE-1 enzymes. 2. RNA and/or DNA are hybridized to the oligonucleotide, for example via RNA polyA tail and known sequences added to transposed DNA. RNA provides template for reverse transcription and Template switch. Captured DNA is ligated to the capture oligo. 3. 1^{st} round of PCR amplification using sequences against the PCR handles, Template switch oligo and transposed MEDS DNA sequence. 4. The PCR amplified product is purified and a second round of PCR performed a. to amplify specifically the DNA from captured RNA, and b. to amplify the captured DNA.

FIG. 4—Tapestation traces show a final library produced for: A. Normal drop-seq using published EZ Macosko—2015 method for performing droplet based sequencing; B. PC drop-seq, as for normal drop-seq but a photocleavable linker is included at the 5′ end of the sequence; and C. PC+HP dual oligo, as described in Example 1.

FIG. 5—UMAP plots showing the number of cells captured by A. Normal drop-seq; B: PC drop-seq; and C: PC+HP dual beads. Each point represents one cell.

FIG. 6—Tapestation trace shows both a DNA library produced from the 5′ capture and an RNA library produced from the 3′ capture. A. Nucleosome phasing is seen following ATAC of HEK293T cells. This is a positive control and confirms that the ATAC protocol generated DNA fragments. B. Shows a final ATAC DNA amplified library following encapsulation and PCR amplification. C. Shows a final post PCR amplified captured RNA product following encapsulation, reverse Transcription and PCR amplification.

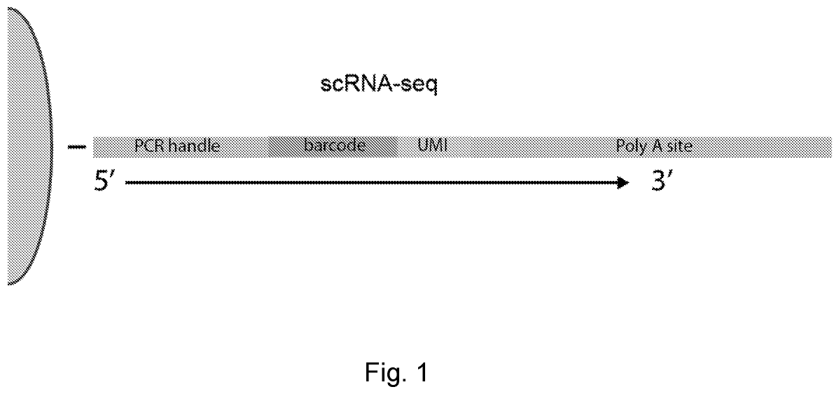

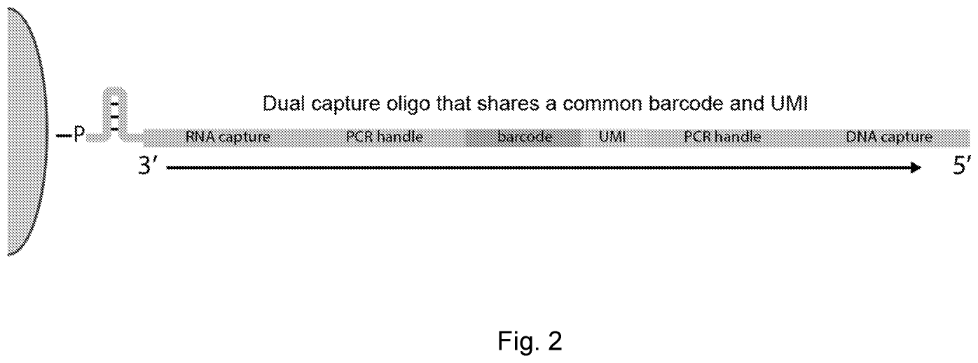

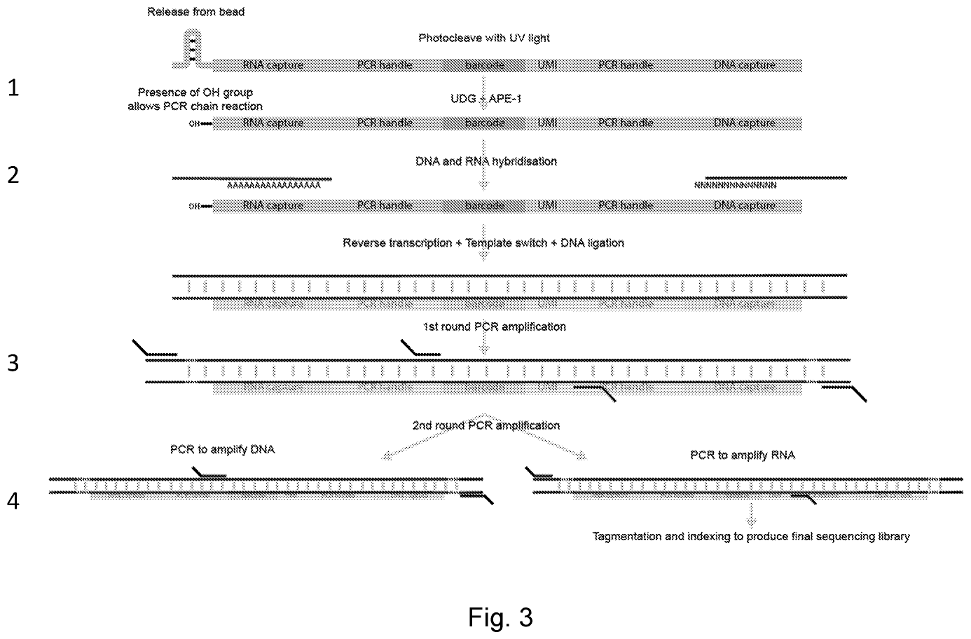

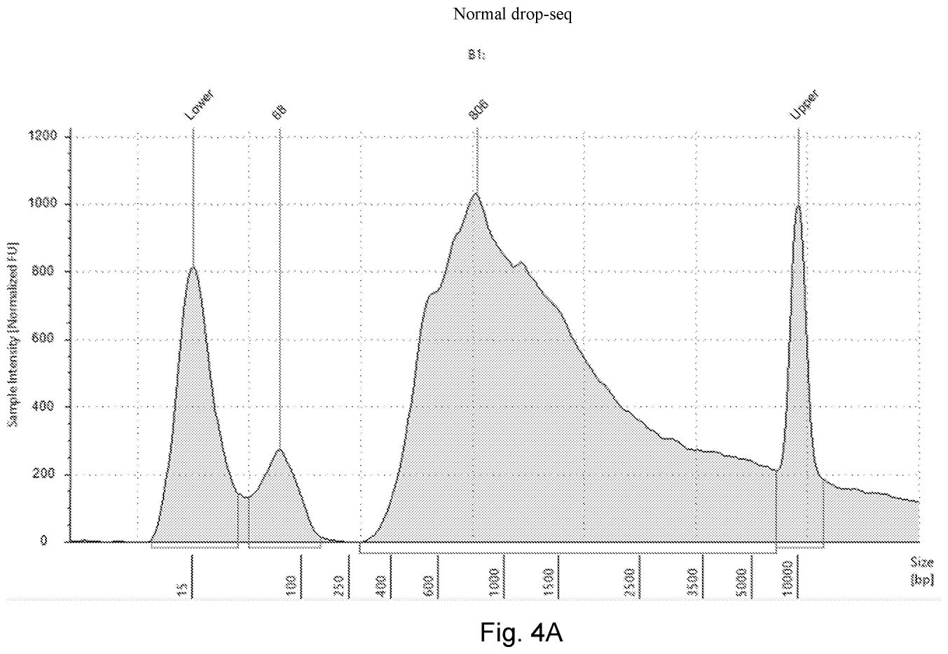

### DETAILED DESCRIPTION OF THE INVENTION

Polynucleotides

The invention relates to arrays of polynucleotides, or micro-particles comprising an array of polynucleotides bound to a micro-bead. The terms “polynucleotide”, “oligonucleotide” or “oligo” may in some cases be used herein interchangeably, and refer to a string of nucleotide monomers in a chain typically linked by phosphodiester bonds. As used herein, a polynucleotide may be a chain of nucleotides of any length, whilst an oligonucleotide typically comprises up to 50 nucleotides. In some cases the polynucleotides of the invention may be at least 50, or at least 56, 60, 70, 80, 90, 100, 110, 120 or 125 and/or up to 130, 140, 160, 180, 200, 225, 250, 275, 300, 350, 400, 500 nucleotides or more in length, for example between 56 and 500 or 400 or 300 or 200 nucleotides in length. The polynucleotides may be DNA (single-stranded DNA) or RNA. Polynucleotides have a chemical orientation defined by the position of the linking carbon in the five-carbon sugar of each consecutive nucleotide in the chain. Polynucleotides may be manufactured by the addition of nucleotides at either the 5′ end (manufacture in a 5′ direction) or the 3′ end (manufacture in a 3′ direction) to elongate the chain. Likewise, sequence elements along the length of a polynucleotide have a sequential order defined by the directionality of the chain of nucleotides that is either 5′ to 3′ or 3′ to 5′.

In some cases, the polynucleotides of the invention comprise the following sequence elements in a 3′ to 5′ direction: (a) a linker that is cleavable to provide a free 3′ hydroxyl group on the polynucleotide after cleavage; (b) a 3′ end analyte capture region; (c) a barcode sequence (BC), wherein the BC of each polynucleotide is the same; and a unique molecular identifier sequence (UMI), wherein the UMI of each polynucleotide is different; and wherein the UMI is 5′ or 3′ to the BC; and (d) optionally a PCR handle sequence. In some cases, the polynucleotides comprise the following sequence elements defined in a 3′ to 5′ direction: (a) a linker that is cleavable to provide a free 3′ hydroxyl group on the polynucleotide after cleavage; (b) a 3′ end analyte capture region; (c) optionally a first PCR handle sequence, (d) a barcode sequence (BC), wherein the BC of each polynucleotide is the same; and a unique molecular identifier sequence (UMI), wherein the UMI of each polynucleotide is different; and wherein the UMI is 5′ or 3′ to the BC; (d) optionally a second PCR handle sequence; and (e) a 5′ end analyte capture region. In other cases, the polynucleotides comprise in a 3′ to 5′ direction: (a) 3′ hydroxyl group; (b) a 3′ end analyte capture region; (c) optionally a first polymerase chain reaction (PCR) handle sequence; (d) a barcode sequence (BC), wherein the barcode sequence of each of the polynucleotides is the same; and a unique molecular identifier sequence (UMI), wherein the UMI of each polynucleotide is different; and wherein the UMI is 5′ or 3′ to the BC; (e) optionally a (second) PCR handle sequence; and (f) optionally a 5′ end analyte capture region.

In some special cases, some of the elements may overlap. In particular, in some cases, the 3′ end analyte capture region may partially or fully overlap with the first PCR handle sequence and/or the 5′ end analyte capture region may partially or fully overlap with the second PCR handle sequence. In some cases, the PCR handle sequence(s) may be positioned fully within the analyte capture region.

In some cases, the polynucleotides may comprise non-nucleotide linking elements (i.e. spacers), for example phosphoramidite spacers, such as 17-O-(4,4′-Dimethoxytrityl)-hexaethyleneglycol, 1-[(2-cyanoethyl)-(N,N-diisopropyl)]-phosphoramidite (HEG). For example, a spacer may be included 3′ to the 3′ analyte capture region, or between the 3′ analyte capture region and the bead. The polynucleotides may also comprise further sequence elements in addition to those defined herein. Typically, however, the 3′ analyte capture region is immediately downstream of the 3′ end hydroxyl group produced on cleavage of the linker. Likewise, the 5′ analyte capture region is typically at the 5′ terminus of the polynucleotide.

Polynucleotides may comprise any combination of natural or canonical nucleotides (i.e., “naturally occurring” or “natural” nucleotides), which include adenosine, guanosine, cytidine, thymidine and uridine. The polynucleotides may also comprise nucleotide analogues. For example, the polynucleotide may include one or more peptide nucleotides, in which the phosphate linkage found in DNA and RNA is replaced by a peptide-like Nr-(2-aminoethyl)glycine. Peptide nucleotides undergo normal Watson-Crick base pairing and hybridize to complementary DNA/RNA with higher affinity and specificity and lower salt-dependency than normal DNA/RNA oligonucleotides and may have increased stability. The polynucleotide may include one or more locked nucleotides (LNA), which comprise a 2′-O-4′-C-methylene bridge and are conformationally restricted. LNA form stable hybrid duplexes with DNA and RNA with increased stability and higher hybrid duplex melting temperatures. The polynucleotide may include one or more Propynyl dU (also known as pdU-CE Phosphoramidite, or 5′-Dimethoxytrityl-5-(1-Propynyl)-2′-deoxyUridine,3′-[(2-cyanoethyl)-(N,N-diisopropyl)]-phosphoramidite). The polynucleotide may include one or more unlocked nucleotides (UNA), which are analogues of ribonucleotides in which the C2′-C3′ bond has been cleaved. UNA form hybrid duplexes with DNA and RNA, but with decreased stability and lower hybrid duplex melting temperatures. LNA and UNA may therefore be used to finely adjust the thermodynamic properties the polynucleotides in which they are incorporated. The polynucleotide may include one or more triazole-linked DNA oligonucleotides, in which one or more of the natural phosphate backbone linkages are replaced with triazole linkages, particularly when click chemistry is used for synthesising the polynucleotide. The polynucleotide may include one or more 2′-O-methoxy-ethyl bases (2′-MOE), such as 2-Methoxyethoxy A, 2-Methoxyethoxy MeC, 2-Methoxyethoxy G and/or 2-Methoxyethoxy T. The polynucleotide may include one or more 2′-O-Methyl RNA bases. The polynucleotide may include one or more 2′-fluoro bases, such as fluoro C, fluoro U, fluoro A, and/or fluoro G. Other specific examples of nucleotide analogues include 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, 5-Methyl dC, 5-Nitroindole, 5-hydroxybutynl-2′-deoxyuridine (Super T) and 8-aza-7-deazaguanosine (Super G). In some cases the polynucleotide may include super T 2,6-Diaminopurine (2-Amino-dA) and/or 5-Methyl dC. The polynucleotide may include one or more biotinylated nucleotides. In some cases the polynucleotide comprises at least two, or at least 3, 4, 5, 6, 7, 8, 9, 10, 12, 14, 16, 18, 20, 25, 30, 35, 40, or up to 2%, 5%, 10%, 15%, 20%, 25%, 30%, 35%, 40% or more nucleotide analogues and/or biotinylated nucleotides, or any one type of nucleotide analogue as described herein.

Linkers

In some cases, the polynucleotides comprise a sequence element that is cleavable to provide a free 3′ hydroxyl group on the polynucleotide after cleavage. Cleavage of the linker may provide a 3′ end analyte capture region, such as a polythymidine for capturing RNA. In some cases the sequence is a linker that is light (e.g. UV light)-sensitive (photocleavage), that is temperature-sensitive or thermolabile (thermocleavage), or that is cleaved on chemical exposure (chemical cleavage).

In other cases, the linker is a sequence element that is cleavable by an enzyme or combination of enzymes to provide a free 3′ hydroxyl group. In one example, the enzymes may be a DNA glycosylase such as a uracil-DNA glycosylase (UDG), and a class I AP endonuclease, such as APE-1. The glycosylase excises a base from the polynucleotide sequence to create an apurinic/apyrimidinic (AP) site, and the endonuclease nicks the phosphodiester backbone leaving a 3′ hydroxyl group. The linker may comprise a uracil. The uracil may be immediately 3′ to the start of the 3′ end analyte capture region, such as a polythymidine region. The linker may comprise a double-stranded region, such as a hairpin structure immediately 3′ to and ending at or including the cleavage site, for example immediately 5′ to a uracil. In another example, the enzyme may be an endonuclease IV, such as the double-strand-specific Escherichia coli endonuclease IV (Nfo) described in Levin et al. (1988, J. Biol. Chem. 263: 8066-8071) and Pirpenburg et al. (2006, PLoS Biol. 4(7): 1115-1121).

In some cases enzymatic cleavage of polynucleotide to provide a free 3′ end hydroxyl group may be more efficient after release of the polynucleotide from bead. Accordingly in some cases, the polynucleotide comprises, in a 3′ to 5′ direction, a first cleavable linker, such as a photocleavable, thermocleavable, chemically cleavable or enzymatically cleavable linker, and a second linker that is cleavable to provide a free 3′ hydroxyl group on the polynucleotide after cleavage. In one example, the polynucleotide may comprise, in a 3′ to 5′ direction, (a) a first cleavable linker, (b) a double stranded region, optionally comprising or followed by a uracil, and (d) the 3′ end analyte capture region, such as a polythymidine. In a specific example, the polynucleotide comprises the sequence HEG-AAAAAAGGCGC-HEG-GCGCCU.

Other examples of enzyme that may be used to provide a 3′ hydroxyl group on polynucleotide after cleavage include restriction enzymes or an esterase. Accordingly, in some cases the linker comprises a suitable restriction site or an ester linkage. If the corresponding restriction enzyme cuts double stranded DNA, then the linker may comprise a double-stranded (or hairpin) region comprising the restriction site. In other cases the linker does not comprise a restriction enzyme cleavage site and/or does not include an ester linkage. Restriction enzymes having longer recognition sites cleave fewer off-target sample polynucleotides. One example restriction enzyme with a seven base pair recognition sequence is SapI (Type IIS restriction enzyme). In some cases the linker may comprise a double stranded region comprising the SapI recognition site (GAAGAGC . . . GCTCTTC) and optionally an adjacent polyT region as the 3′ analyte capture region. For example, the sequence GAAGAGCT-HEG-AGCTCTTC could replace the region between the polyT and polyA in the polynucleotide described herein in Example 1, with or without including the PCLinker between the double-stranded region and the bead. The restriction site is outside of the recognition site and would cut within the polyT region in the example above, leaving a 3′ polyT region with a free 3′ hydroxyl group.

BspQI, an isoschizomer of SapI, may also be used. However, BspQI needs a higher incubation temperature than SapI and may be less buffer tolerant.

Example restriction enzymes having a six base pair recognition sequence include BspHI, BspEI, MmeI, NruI, XbaI, BclI, FspI, MscI, BsrGI, PsiI-v2, BstBI and DraI (well-known type II restriction enzymes) and BbsI, BciVI, BmrI, BsaI, Earl & Esp3I (well-known type IIs restriction enzymes). The known recognition and restriction sites of these restriction enzymes may be included in the linker, in a double-stranded/hairpin region as needed, optionally adjacent to a polyT region as the 3′ analyte capture region. For example the BsrGI recognition sequence is T/GTACA . . . T/GTACA. In one example, the sequence T/GTACAT-HEG-AT/GTACA could replace the region between the polyT and polyA in the polynucleotide described herein in Example 1, with or without including the PCLinker between the double-stranded region and the bead.

Analyte Capture Regions

The analyte capture region(s) may be any nucleotide sequence suitable for capturing analyte in a sample. In some cases the analyte(s) may be biological analytes or may be selected from polynucleotides such as DNA, cDNA and/or RNA, or from polynucleotides, oligonucleotides, DNA, RNA, mRNA, proteins, polypeptides and/or peptides, cell surface receptors or cells. In some cases the analytes may additionally be selected from amino acids, metal ions, inorganic salts, polymers, nucleotides, oligonucleotides, polynucleotides, dyes, bleaches, pharmaceuticals, diagnostic agents, recreational drugs, explosives and/or environmental pollutants. Such analytes may be captured, for example, by an aptamer.

Typically the analyte capture region(s) sequence may be at least 10, or at least 15, 20, 25 or 30 nucleotides in length, such as from about 15 to about 50, from about 20 to about 40 or from about 25 to about 35 nucleotides. In some cases, the analyte capture region(s) may comprise one or more nucleotide analogues, such as analogues described herein, that form double-stranded hybrids with higher stability than natural nucleotides. In this case, the analyte capture region(s) could be shorter, such as at least 3, 4, 5, 6, 8 or 9, for example between 3 and 50, or 40 or 30 or 20 nucleotides in length, provided that the analyte capture region(s) was capable of hybridizing to target analyte such that analyte sequence can be amplified as described herein. One or both analyte capture regions may include nucleotide analogues as described herein.

In some cases an analyte capture region may be a DNA capture region, an RNA or mRNA capture region, or a polypeptide capture region.

In one example, an analyte capture region, particularly the 3′ analyte capture region may be a polythymidine. Polythymidine may hybridise to and capture any polynucleotide in the sample that comprises a suitable polyadenosine, such as polyadenylated mRNA. Typically the polythymidine may be at least 10, or at least 15, 20, 25 or 30 thymidines in length, such as from about 15 to about 50, from about 20 to about 40 or from about 25 to about 35 thymidines. When the polynucleotide is attached to bead, the polythymidine may be immediately to the 3′ side of the cleavage site of the linker that provides the free 3′hydroxyl group after cleavage. When polynucleotide is not attached to bead, the polythymidine comprises the hydroxyl group at the free 3′ end of the polynucleotide.

In other cases, the analyte capture region(s) may comprise or consist of an aptamer. Aptamers can be produced using SELEX (Stoltenburg, R. et al., (2007), Biomolecular Engineering 24, p381-403; Tuerk, C. et al., Science 249, p505-510; Bock, L. C. et al., (1992), Nature 355, p564-566) or NON-SELEX (Berezovski, M. et al. (2006), Journal of the American Chemical Society 128, p1410-1411). Typically, an aptamer may be at least 15 nucleotides in length, such as from about 15 to about 50, from about 20 to about 40 or from about 25 to about 30 or nucleotides in length. An aptamer may bind to analyte such as small molecules, proteins, nucleic acids or cells. Aptamers may be designed or selected to bind to pre-determined target analyte(s). In one example, the aptamer may bind to a Coronaviridae protein or SARS-CoV-2 protein, such as any of the SARS-CoV-2 structural protein sequences provided herein.

In some cases, an analyte capture region(s) may comprise or consist of a biotinylated nucleotide sequence. Nucleotides or polynucleotides may be biotinylated using methods known in the art. Typically the biotinylated sequence may be at least 10, or at least 15, 20, 25 or 30 nucleotides in length, such as from about 15 to about 50, from about 20 to about 40 or from about 25 to about 35 nucleotides. A biotinylated capture region may be used to capture any suitable target analyte comprising streptavidin or avidin.

In some cases, the analyte capture region(s) may comprise or consist of a nucleotide sequence designed to hybridise to a complementary sequence in a target polynucleotide analyte. In some cases the capture region is for capturing/hybridising to transposed DNA. In this case an analyte capture region may comprise or consist of a sequence that is complementary to transposed DNA in a sample, for example to a transposed MEDS DNA sequence. In some cases, the sequence may be gene or transcript-specific, such as a polynucleotide sequence that is complementary to, or at least 80%, 85%, 90%, 95%, 98% or 99% complementary to, a viral sequence, a bacterial sequence or a sequence associated with a disease or disorder, such as a sequence from a cancer-associated antigen or a neoantigen. In some cases the analyte capture region(s) may hybridise to a nucleotide sequence that encodes a part of a Coronaviridae protein or SARS-CoV-2 protein, such as any of the SARS-CoV-2 structural protein sequences provided herein.

In other cases, the sequence may be designed to capture a polynucleotide tag added to analyte of interest.

In some cases, 5′ analyte capture region may be absent or may comprise any of the characteristics described herein for the 3′ analyte capture region. Typically, however, the 5′ analyte capture region does not consist of a polythimidine sequence because mRNA hybridised to polythimidine at the 5′ analyte capture region cannot be converted to cDNA by reverse transcription from the 5′ end.

In some cases the 3′ and 5′ analyte capture regions may be for binding the same type of analyte. For example, both regions may be DNA analyte capture regions, or both ends may be biotinylated and bind to analyte comprising streptavidin or avidin, or both regions may be protein capture regions, and/or both ends may comprise an aptamer. In some cases, the 3′ and 5′ analyte capture regions may be for binding different types of analyte. For example, in some cases the 3′ analyte capture region be for binding RNA, for example the 3′ analyte capture region be a polythimidine, and the 5′ analyte capture region may be for binding DNA or protein, such as any types of DNA or protein described herein. In a different example, both analyte capture regions may comprise different sequences for hybridising to complementary sequence in different polynucleotide analytes.

Barcode Sequences (BC)

The barcode sequence identifies analyte that was captured by polynucleotides that were initially isolated as an array of polynucleotides, for example, the array of polynucleotides associated with the same micro-bead or micro-particle, and distinguishes analyte that was captured by polynucleotides initially associated with different microbeads, micro-particles or arrays of polynucleotides. Accordingly, the barcode sequence of each polynucleotide in the array, or associated with a particular micro-bead or micro-particle, is the same as the barcode sequence in every other polynucleotide in the array, or associated with the same micro-bead or micro-particle. Barcode sequences may be added to polynucleotides being synthesised on a micro-bead using a typical split-and-pool method as known in the art. A pool of micro-particles is split into four separate reactions, each comprising a different one of the four nucleotide bases to be added at the 5′ end of each polynucleotide, then re-pooled. Repeat split-pool cycles creates a pool of micro-particles with a large diversity of polynucleotide barcode sequences. For example, 12 cycles results in 412 (16,777,216) possible barcode sequences. Hence, in a typical experiment using ˜150,000 beads, the polynucleotides of every bead will have a barcode sequence that is different from essentially every other bead. In general, experiments that need more uniquely identifiable beads require a greater barcode sequence diversity and hence longer barcode sequences. A typical barcode sequence may be at least 6, 7, 8, 9, 10, 11 or 12 nucleotides in length, for example, about 10 to 14, or 11 to 13 nucleotides in length.

Unique Molecular Identifier Sequence (UMI)

The unique molecular identifier sequence identifies analyte that was captured by a single polynucleotide and distinguishes analyte that was captured by different polynucleotides in the same array or associated with the same microparticle or microbead. UMIs may be used to digitally count analyte and detect duplicate sequences derived from a single capture event. Accordingly, in some cases the UMI of each polynucleotide in the array, or associated with a particular microbead or microparticle, may be different from the UMI of essentially every other polynucleotide in the array or associated with the same particular microbead or microparticle. In other cases, the UMI diversity is sufficient that essentially every capture polynucleotide that captures the same species of capture analyte, e.g. mRNAs having the same sequence, has a different UMI, or that the repeat UMIs are sufficiently infrequent as not to interfere with digital counting. UMIs may be synthesised using multiple rounds of degenerate synthesis in the presence of all four DNA bases, as known in the art. For a typical bead, eight rounds generates a UMI diversity of 4⁸ (65,536). This may in some cases be sufficient to ensure that the UMI of every polynucleotide associated with each bead is different from the UMIs on essentially every other polynucleotide associated with the same bead. In other cases, it may be sufficient to design the length and diversity of the UMIs such that essentially every capture polynucleotide that captures the same species of capture analyte, e.g. mRNAs having the same sequence, has a different UMI, or that the repeat UMIs are sufficiently infrequent as not to interfere with digital counting. Therefore, shorter or longer UMI sequences could be used for beads comprising fewer or more polynucleotides, or for samples that include different quantities or diversity of capture analytes. Accordingly, a typical UMI may be at least 6, 7, or 8 nucleotides in length, for example, about 6 to 10, or 7 to 9 nucleotides in length. The skilled person is able to select an appropriate length of UMI depending on the experiment.

PCR Handle Sequences

A PCR handle sequence hybridizes to PCR oligonucleotide primers during a PCR reaction. Typically a PCR handle sequence may be at least 15, 16, 17, 18, 19 or 20 nucleotides in length and/or up to and 21, 22, 23, 24, 25, 30 or 35 nucleotides in length, for example about 15 to 30, or 18 to 25 nucleotides. In some cases shorter PCR handle sequences may be used. In some cases, the PCR handle sequence(s) may comprise one or more nucleotide analogues, such as analogues described herein, that form double-stranded hybrids with higher stability than natural nucleotides. In this case, the PCR handle sequence(s) could be shorter, such as at least 3, 4, 5, 6, 8, 9, 10, 11, 12, 13 or 14 nucleotides in length, provided that the PCR handle sequence(s) was capable of hybridizing to PCR oligo as described herein. One or both PCR handle sequence(s) may include nucleotide analogues as described herein.

In some cases the PCR handle sequences of each polynucleotide in an array, or associated with the same microbead or microparticle, may be the same. In other cases, difference sequences may be used. In some cases, the polynucleotides may have a first and a second PCR handle sequence as described herein. The first and second PCR handle sequences may, in some cases, have the same or complementary sequences. In other cases, different or non-complementary sequences are used. In some cases, the invention relates to a plurality of microbeads or microparticles. In some cases, the (first and/or second) PCR handle sequences of the polynucleotides of each of the microbeads or microparticles are the same as the (first and/or second) PCR handle sequences of essentially each other microbead or microparticle. In some cases one or more of the PCR handle sequences comprises or consists of one of the following sequences:

5′-GTGGTATCAACGCAGAGTAC-3′
5′-GTCCGAGCGTAGGTTATCCG-3′

These PCR handles could also be compatible with standard Illumina sequencing to eliminate the need for custom read 1 sequencing primers to read BC/UMI.

In some cases, one or both PCR handles may be omitted. For example, long-read sequencing technologies such as Nanopore or Pacbio may be used to directly sequence DNA or RNA without the need for PCR amplification. As such, analytes captured by each polynucleotide may be converted, for example, into a Nanopore library for direct sequencing. In another example, an adapter comprising a PCR handle sequence may be ligated to analytes subsequent to their capture by polynucleotide, and PCR performed to amplify the analyte. In a third example, the known sequence(s) of the 3′ and/or 5′ analyte capture regions, or a portion thereof, may be used as PCR handle sequences. Hence, in this case there is no need for separate sequences for analyte capture and PCR priming.

Micro-Particles

In some embodiments the invention relates to micro-particles. The micro-particles comprise a micro-bead and an array of polynucleotides (micro-particles) as described herein. Microbeads are typically less than 500 μm, or less than 400 μm, 300 μm or 200 μm in diameter, for example, between 10 and 500 μm, 20 and 400 μm, 40 and 300 μm, or 50 and 200 μm. Typically a micro-bead is approximately spherical or sphere-like. Micro-beads with surface-attached polynucleotides are well-known in the art and may be made from, for example, a biocompatible polymer such as polystyrene, polyacrylamide or hydroxylated methacrylic polymer, or from controlled pore glass. The micro-particles described herein may comprise a micro-bead as described herein with an array of polynucleotides as described herein, wherein each polynucleotide in the array is attached to the bead at the 3′ end of the polynucleotide. The 5′ end is typically free in solution.

Dissolvable beads and hydrogel beads have also been described and are encompassed in the present disclosure. Dissolvable beads may, for example, be made from crosslinked acrylamide with disulfide bridges that are cleaved with dithiothreitol. An array of polynucleotides may be embedded in the bead matrix and released when the bead is dissolved. A micro-particle comprising a micro-bead and an array of polynucleotides bound to the micro-bead may be a typical micro-bead with surface bound polynucleotide or a dissolvable bead with embedded polynucleotide.

Polynucleotides may be synthesised on the bead using methods known in the art. For example, the phosphoramidite method may be used, in which one nucleotide is added per synthesis cycle. The barcode sequence and UMI sequences are typically generated as described elsewhere herein, Longer sequence elements may in some cases be added using enzymatic ligation methods, such as using DNA ligase, chemical ligation methods, such as phosphoramidate ligation, and/or click chemistry ligation methods, such as the azide-alkyne cycloaddition reaction. Suitable methods are known in the art.

In some cases, the invention relates to a plurality of micro-particles as described herein. Typically the number of micro-particles is sufficient to conduct a typical experiment or to generate one or more libraries. Typically the plurality of micro-particles comprises at least 1000, or at least 10,000, or 50,000, or 100,000, or 150,000, for example between 1000, or 10,000, or 50,000, or 100,000, or 150,000 and 10⁴, or between 10,000 and 10⁶, or between 50,000 and 600,000, or between 50,000 and 400,000, or between 10,000 or 100,000 and 300,000 micro-particles, or between 1000 and 600,000, or between 50,000 and 400,000, or between 10,000 and 300,000 micro-particles. The set of polynucleotides associated with each micro-particle may have a different shared barcode sequence from the polynucleotides associated with essentially each of the other micro-particle. Hence, the analytes that are captured by the set of polynucleotides associated with each micro-particle can be distinguished. The potential diversity of barcode sequences is dependent on the barcode sequence length, as described above. The barcode sequence will typically be long enough that the potential sequence diversity is well in excess of the plurality of micro-particles, typically at least 50× or 100× in excess, or least 10, 20, 50, 100, 200 or 500 times in excess. For example, for a typical experiment using a pool of approximately 150,000 micro-particles, a 12 nucleotide barcode sequence provides a potential diversity of 4¹² (16,777,216) different sequences, which provides an excess of barcode sequences to beads of >100×.

Array of Polynucleotides

In some cases the invention relates to an isolated set or array of polynucleotides as described herein. Each polynucleotide in the array is characterised in comprising the sequence elements and orientation described herein. The polynucleotides of the array all share the same barcode sequence as described herein. Each of the polynucleotides of the array may also have a different UMI sequence as described herein. The barcode sequence may be 3′ or 5′ to the UMI sequence. Each of the polynucleotides also comprises the other sequence elements as described herein 3′ and 5′ to the barcode and UMI sequences. This arrangement ensures that sequencing libraries generated from analytes bound to one or both ends of the polynucleotides will always include a barcode sequence that identifies the array of polynucleotides and a UMI sequence that identifies the individual polynucleotide.

The array of polynucleotides may be bound to a micro-bead as described herein, or may otherwise be isolated from other polynucleotides having the same set of sequence elements and directionality, but a different barcode sequence. For example, the array of polynucleotides may be the set of polynucleotides released from a micro-bead as described herein by cleavage of the linker to provide a set of polynucleotides each having a free 3′ hydroxyl group. Typically the micro-particle is isolated from other similar micro-particles, for example in a fluidic compartment as described herein, prior to polynucleotide cleavage, such that the array of polynucleotides is isolated from other polynucleotides having the same sequence elements and directionality.

The array typically comprises at least 200, or at least 1000, 10,000, 100,000, 1,000,000, 10⁷, 10⁸, 10⁹ or 10¹⁰ polynucleotides, for example between 200 and 10¹² or between 10⁵ and 10¹¹ polynucleotides.

The potential diversity of UMI sequences is dependent on the UMI sequence length, as described above. The UMI sequence will in some cases typically be long enough that the potential sequence diversity is well in excess of the number of polynucleotides in the array, or bound to the micro-bead, typically at least 50× or 100× in excess. For example, for a typical micro-particle comprising a micro-bead and an array of approximately 500 bound polynucleotides, an 8 nucleotide UMI sequence provides a potential diversity of 4⁸(65,536) different sequences, which provides an excess of UMI sequences to polynucleotides of >100×.

In some cases, all of the polynucleotides of the array may have the same 3′ and/or 5′ analyte capture regions. In other cases different 3′ and/or 5′ capture regions may be used. For example, the 3′ and/or 5′ capture regions of the array could comprise different aptamers or may have different sequences for hybridising to different sequences in DNA analytes. In some cases the 3′ and/or 5′ analyte capture regions may consist of at least two, or at least 3, 4, 5, 10, 20, 50, 100 or 200 different polynucleotide sequences. In some cases the 3′ or 5′ analyte capture region may be the same in each polynucleotide of the array, but the other analyte capture region may have different sequences amongst the polynucleotides of the array.

In some cases, some of the polynucleotides in the array may be bound to analyte at the 3′ and/or 5′ end as described herein.

In some cases the bound analyte comprises a polynucleotide sequence that is complementary to, or at least 80%, 85%, 90% 95%, 98%, 99% complementary to, the polynucleotide sequence of the analyte capture region, and the complementary polynucleotide sequence of the analyte sequence is hybridised to the anayte capture region.

In some cases, at least about 0.000005%, or 0.00001%, or 0.00005%, or 0.0001%, or 0.0005%, or at least about 0.001%, or 0.005%, or 0.01% or 0.05% or 0.1% of polynucleotides in the array may be bound to or hybridised to analyte. Where the polynucleotides comprise both a 3′ analyte capture region and a 5′ analyte capture region, some of the polynucleotides may bind or hybridise to one analyte via the 5′ analyte capture region and to a second analyte via the 5′ analyte capture region. In some cases, at least about 0.0000005%, 0.000001%, or 0.000005%, or 0.00001%, or 0.00005%, or 0.00001%, or at least about 0.0001%, or 0.001%, or 0.010%, or 0.10% of polynucleotides in the array may be bound to or hybridised to analyte at both ends of the polynucleotide.

In some cases the same type of analyte may be bound or hybridised at both ends of polynucleotide. For example, both the 3′ analyte capture region and the 5′ analyte capture region may be bound to DNA, or to protein. In some cases, analyte may bind to the 3′ and 5′ analyte capture regions sequentially. In some cases, for example, mRNA may bind to a 3′ polythymidine analyte capture region and reverse transcription reaction produces an RNA/cDNA hybrid at the 3′ end. The 5′ analyte capture region may subsequently capture a different analyte.

Analyte Capture and Library Generation

In some embodiments, the invention provides a method for capturing analyte in a sample. The method comprises contacting the sample with a micro-particle or an array of polynucleotides as described herein and allowing analytes in the sample to bind to the 3′ and/or 5′ analyte capture regions of the array of polynucleotides.

In some cases, the analytes are from a single cell, such as a bacterial cell, or single cell nucleus, single cell vesicle, such as an exosome or mini-vesicle, or other compartment enclosed by a lipid membrane. In some cases the method comprises isolating the single cell, nucleus, vesicle or other compartment, or a lysate thereof, and contacting the isolated cell, nucleus, vesicle, compartment or lysate with a single micro-particle or array of polynucleotides as described herein.

In some cases, the analytes are from a sample comprising a plurality of cells (such as bacterial, prokaryotic or eukaryotic cells), cell nuclei, vesicles or other compartment enclosed by a lipid membrane, or a two or three dimensional sample such as a tissue sample. Analytes from different cells, cell nuclei, vesicles or other compartments or from different spatial positions in the sample may be captured by separate arrays of polynucleotides as described herein. The method may in some cases include preparing a single cell or single cell nuclei or vesicle suspension from the sample and/or isolating single cells, nuclei, vesicles or lysates thereof in separate compartments. A single cell, nuclei, vesicle or cell/nuclei/vesicle lysate of the sample and a single micro-particle may be isolated in each of a plurality of separate compartments. The polynucleotide array of each micro-particle may have a different barcode sequence from essentially each other micro-particle in each separate compartment. Typically, there may be at least 500, or at least 5,000, or 25,000, or 50,000, or 100,000, for example between 500 and 500,000, or between 2,000 and 200,000, or between 20,000 and 100,000 separate compartments, each comprising a single cell, nuclei or cell/nuclei lysate and a single micro-particle or array of polynucleotides as described herein.

Micro-particles or arrays of polynucleotides may be contacted with sample or analyte in a compartment, typically a fluidic compartment. For example, the compartment may be a well, such as a well in a multi-well plate or microplate or slide, a discrete site on a microfluidic chip, or a (micro-)droplet, which may be formed in an oil emulsion. Such compartments may be made using a microfluidics device as known in the art. In some cases, the sample, a micro-particle, an array of polynucleotides, a cell, cell nuclei or cell/nuclei lysate may be encapsulated or co-encapsulated in a fluidic compartment.

The cell and/or cell nucleus membrane(s) may be lysed, before or after contact with the micro-particle or array of polynucleotides. In some cases the cell and/or nucleus may be lysed and target polynucleotides may be amplified, for example by PCR, before being brought into contact with the microparticle or array of polynucleotides. The analytes that bind to the 3′ and/or 5′ analyte capture regions may then be include the PCR products.

The linker of the polypeptides may be cleaved from the bead of the micro-particle before or after contact with the cell, nucleus or lysate, for example by exposing the micro-particle to UV light or heat, or contacting the micro-particle with appropriate chemicals or enzyme(s), such as the enzyme(s) described herein. In some cases, it may be convenient to include the micro-particle or array or polynucleotides in cell/membrane lysis buffer, and/or the cell, nucleus or lysate in a buffer comprising agents needed for cleavage of the linker, such that mixing the two buffers exposes cell/nucleus to lysis buffer and/or micro-particle to the chemical milieu for linker cleavage, as well as bringing the array of polynucleotides into contact with analyte from the cell or nuclei. For example, a microfluidics device may be used to join two aqueous flows into discrete microfluidic droplets. One flow may comprise a single cell or single cell nuclei suspension in cell buffer and optionally chemicals or enzymes needed to cleave the polynucleotide linker. The other flow may comprise a suspension of micro-particles as described herein, optionally in cell/membrane lysis buffer. Some of the droplets that are formed comprise both a cell or nuclei and a micro-particle, resulting in contact between the analytes and the array of polynucleotides.

Other components needed for downstream reactions and processes may be included in the cell/nucleus buffer and micro-particle/lysis buffer. In some cases, the cell/nucleus buffer may comprise template switch oligonucleotides. In some cases, the micro-particle/lysis buffer may comprise reverse transcriptase, in particular when the 3′ analyte capture region of the polynucleotides is an RNA capture region, such as a polythymidine.

RNA in the sample may bind to the 3′ RNA capture regions of the polynucleotides and reverse transcription using the bound RNA as template provides an RNA/cDNA hybrid at the 3′ end of the polynucleotide. Template switch oligonucleotides may be added at the end of the RNA/cDNA hybrid. The template switch oligonucleotides and the PCR handle sequence 5′ to the BC and UMI sequences provide a pair of PCR handle sequences for PCR amplification of the cDNA/RNA hybrid. PCR amplification may use a pair of oligonucleotide primers that hybridise to the template switch oligonucleotide sequence and the PCR handle sequence 5′ to the BC an UMI sequences.

DNA in the sample may bind to a 5′ and/or 3′ DNA capture region of the polynucleotides. DNA captured at the 5′ end may be amplified using oligonucleotide primers that hybridise to a PCR handle sequence on the captured DNA and the complement of the PCR handle sequence 3′ to the BC and UMI sequences. DNA captured at the 3′ end may be amplified using oligonucleotide primers that hybridise to a PCR handle sequence on the captured DNA and the PCR handle sequence 5′ to the BC and UMI sequences.

When the array polynucleotides comprise both 3′ and 5′ analyte capture regions, a first round of PCR amplification using all of the relevant PCR oligonucleotide primers will generate PCR products comprising both the barcode and UMI sequences and polynucleotide sequence from polynucleotide analyte bound to the 3′ or 5′ analyte capture regions. A third PCR product comprising the barcode and UMI sequences, but not any sequence from bound analytes, will also be generated from the pair of primers that hybridise one to the PCR handle sequence 5′ to the barcode and UMI sequences and the other to the complement to the PCR handle sequence 3′ to the barcode and UMI sequences. One or more further rounds of PCR amplification using only one or the other pair of PCR oligonucleotide primers described above eliminates this additional PCR product from further amplification.

After amplification, the PCR products may be sequenced using methods known in the art. Barcoding means that different compartments containing different polynucleotide arrays bound to analyte, or reaction products thereof, may be merged after capture. For example, the different compartments, such as droplets, may be merged after reverse transcription and/or before PCR. Downstream processes may then be carried out in bulk.

PCR product sequences having different barcodes can be digitally assigned to different samples, cells or cell nuclei. Analytes can be digitally counted by using the UMI sequences to identify duplicates derived from the same capture event.

The methods described herein can be used to generate libraries corresponding to the analytes that bind to the 3′ and/or 5′ analyte capture regions of the polynucleotides described herein. A library may correspond to the analytes from a single cell, cell nuclei or other sample that is contacted with a single micro-particle or array of polynucleotides as described herein, or may correspond to the analytes from a plurality of single cell, cell nuclei or other samples. In some cases a first library may be generated from the analytes bound to the 3′ analyte capture regions, and a second library may be generated from the analytes bound to the 5′ analyte capture regions, particularly when the 3′ and 5′ analyte capture regions capture different types of analyte as described herein. In this case, barcoding the polynucleotides of each micro-particle or array of polynucleotides as described herein allows matching of the libraries or parts of each library that correspond to analytes from the same cell, nuclei or sample. For example, captured RNA and DNA analytes (or any other two or more analyte types described herein) that are captured by the same array of polynucleotides contacted with the same cell, nucleus or sample may be digitally matched and distinguished from RNA and DNA (or other analytes) captured by other arrays of polynucleotides contacted with other cells, nuclei or samples analysed in the same experiment.

### EXAMPLES

### Example 1—Exemplary Protocol for 3′ End mRNA Capture and Library Generation from Single Cells

Reagents and buffers:

Reagent

Cell buffer
PBS
BSA
Template switch oligo (TSO)
Lysis buffer
RT buffer (5X)
Ficoll PM-400
Triton X-114
Maxima H-Rev transcriptase
ATP 100 mM
dNTPs
RNAse Inhibitor
QX200± ™ Droplet Generation Oil for EvaGreen
Perflurooctanol (PFO)
PCR mix
Kapa HiFi polymerase
PCR primer
AMPure beads
BioAnalyzer High Sensitivity D5000 tape
BioAnalyzer High Sensitivity D1000 tape
Nextera XT DNA Library Preperation kit
70 μm Flowmi filter
Neubauer Improved Haemocytometer
Fuchs-Rosenthal Haemocytometer (plastic)

Reagents and buffers:

Lysis buffer is preferably made fresh. Cell buffer is prepared fresh and filtered through a 0.2 μm syringe filter.

| Concentration/volume | Component |
|---|---|
| Lysis buffer | |

| | | |
|---|---|---|
| 100 | μL | 5χ RT buffer |
| 80 | μL | Ficoll PM-400 |
| 1.25 | μL | Triton-X114 |
| 20 | μL | dNTP |
| 20 | μL | RT enzyme |
| 5 | μL | RNAse Inhibitor |
| 2 | μL | ATP |
| 21.75 | μL | H₂O |
| 250 | μL | |

| | |
|---|---|
| Cell buffer | |
| 1X | PBS |
| 0.01% | BSA |

| | | |
|---|---|---|
| 15 | μL | TSO |
| 10 | μL | UDG |
| 10 | μL | APE-1 |

### Primers

| Name | Sequence |
|---|---|
| Template | 5′-AAGCAGTGGTATCAACGCAGAGTGAATrGrGrG |
| switch | |
| oligo | |
| SMART PCR | 5′-AAGCAGTGGTATCAACGCAGAGT |
| PRIMER | |
| New-P5- | 5′-AATGATACGGCGACCACCGAGATCTACACGCCT |
| SMART | GTCCGCGGAAGCAGTGGTATCAACGCAGAGT* |
| PCR | A*C |
| hybrid | |
| oligo | |
| Read1 | 5′- |
| Custom | GCCTGTCCGCGGAAGCAGTGGTATCAACGCAG |
| primer | AGTAC |

Capture beads:

5′-TCGGACCGTTCGTCGGTGGTATCAACGCAGAGTACJJJJJJJJJJJJ
NNNNNNGTCCGAGCGTAGGTTATCCGTTTTTTTTTTTTTTTTTTTTTTTT
TTTTTTUCCGCG-HEG-GCGGAAAAAA-HEG-PCLinker-T-Bead-3′

rG=ribonucleic acid Guanine
* =Phosphorothioated DNA nucleotides
HEG=Spacer-CE Phosphoramidite 18 (aka spacer 18)
N=unique molecular identifier (UMI) nucleotide
J=barcode (BC) sequence nucleotide
PCLinker=PC Linker-CE Phosphoramidite (3-(4,4′-Dimethoxytrityl)-1-(2-nitrophenyl)-propane-1,3-diol-[2-cyanoethyl-(N,N-diisopropyl)]-phosphoramidite)

Beads in dry resin form require washing with 20 mL of 100% ethanol and then washing with 30 ml of TE-TW and resuspension in 20 mL of TE-TW. Bead numbers are determined using a Fuchs-Rosenthal Haemocytometer (plastic). Beads are stored at +4° C. for 6 months or longer.

### Encapsulation

Power up the Nadia instrument with the desired chip and place it on the instrument
Ensure the locating pins on the instrument slot into corresponding cut-outs in the cartridge
Remove the gasket from the chip.
Follow on-screen instructions and using a P1000 pipette or a powered aspirator/dispenser, load 3 ml of emulsion oil as required into the oil reservoir
Re-apply the gasket making sure the gasket fits over the 4 pins in the corners.
Press Next to begin pre-cooling
   Bead preparation:
Preferably use low-retention pipette tips to minimise bead loss.
Prepare 1 ml of lysis buffer and place on ice
Transfer 155,000 beads from the stock suspension into a new tube and spin down at
1,000 g for 1 min, discard the supernatant using a P200 pipette leaving only the remaining pelleted beads
Resuspend the beads in 250 μl of cold Lysis buffer and store on ice until needed
   Cell preparation:
Prepare the cell buffer according to the buffer instructions. The TSO is added to the cell buffer as it is required following in droplet reverse transcription (RT).
Prepare the cells in a single cell suspension as desired. The protocol below uses HEK or 3T3 cells.
Trypsinise cells for 5 mins using TrypLE
Spin down cells at 300 g for 5 mins
Resuspend the cells in 1 ml of 1×PBS
Spin down cells at 300 g for 3 mins
Remove supernatant and resuspend in Cell buffer (minus TSO)
Sieve the cell through a 70 μm (you can also use 40 μm if required) and count in a cell counter or haemocytometer.
Spin down cells and resuspend in cold Cell Buffer (310 cells/μl). If mixed species then take 38,250 cells from each for the total 77,500 cells and pool into a final volume of 250 μl. Alternatively 77,500 single cells of the same cell type can be used. Place cells on ice until needed.
   Run cells on the Nadia instrument:
Press ‘Next’ on the Nadia screen to open the lid
Follow on-screen instructions to load the beads into the blue flashing wells:
   Carefully mix the beads about 10×by using standard P200 pipette tips
   Set the pipette to 125 μl and load 250 μl of bead suspension using low retention tips
Next, cells are loaded into the orange flashing wells:
   Carefully resuspend the cells using a P200
   Set the pipette to 125 μl and load 250 μl of cells.
Replace the gasket and close the lid
Start the run. The Nadia instrument will take around 25 mins to finish for a cell run and 1.5 hours for a single-nuclei run.
When the run is complete an emulsion will be present in the output well of the chip. The emulsion should be creamy white in appearance and will be floating atop a layer of oil.
Using a P1000 pipette tip remove as much oil from the well as possible but be careful not to remove beads.
With some residual oil in the well remaining, pipette the oil and emulsion into a 1.5 ml low bind Eppendorf (from now on use exclusively low bind tubes).
   UV irradiate the beads:

Irradiate the beads using UV light so that the photocleavable linker breaks the connection between the bead and the oligonucleotide. The oligo is then free in solution and can bind RNA.
Open the cap of the 1.5 ml Eppendorf and place UVP dual tube handheld UV lamp above the tubes
Turn on the UV lamp and set it to long wave setting
UV irradiation is complete after 10 min.
   Create free 3′ OH:
Incubate the 1.5 ml Eppendorf at 37° C. for 1 hr
   Reverse transcription:
Incubate the tube at room temperature (22° C.) for 30 mins
Incubate the tube at 42° C. for 1.5 hours
   Breaking emulsion and clean-up:
Add 100 μl of PFO to the sample then invert tubes three times
Spin down sample at 1000 g for 1 min
Collect the aqueous phase (about 250 μl) and add to a new 1.5 ml tube.
Spin down at 1000 g for 1 min again and remove sample to new 1.5 ml tube to remove residual beads
   Purification of cDNA:
Vortex bottle of AMPPure beads to mix
Add 1×(1:1 beads to sample ratio, usually 250 μl) of AmpPure XP beads to each tube of sample.
Perform the clean-up following manufacturers protocol
Elute in 25 μl of H₂O
   PCR with kapa HiFi:

Following addition of the SMART adapters following RT (using TSO) in the previous step, perform PCR amplification of the cDNA using primers against the SMART sequence.
Defrost master mix and SMART PCR primer
Place 24.6 μl of cDNA into a PCR tube and then add the following components:

| Volume (μl) | Component |
|---|---|
| 24.6 | cDNA |
| 0.4 | 100 μM SMART PCR primer |
| 25 | 2X Kapa HiFi MM |

Then proceed to PCR:

| Cycles | T [° C.] | Time | |
|---|---|---|---|
| | 95 | 3 | min |
| 4X | 98 | 20 | s |
| | 65 | 45 | s |
| | 72 | 3 | min |
| 9X | 98 | 20 | s |
| | 67 | 20 | s |
| | 72 | 3 | min |
| | 72 | 5 | mins |

| | |
|---|---|
| 4 | ∞ |

Purification of cDNA and evaluate on Tapestation:
Vortex bottle of AMPPure beads to mix
Add 0.6×(0.6:1 beads to sample ratio, usually 250 μl) of AmpPure XP beads to each tube of sample.
Perform the clean-up following manufacturers protocol
Elute in 20 μl of H₂O
Run 2 μl of sample on a d5000 high sensitivity tape and run on Tapestation according to manufacturer's instructions.
The cDNA may produce a spiky or smooth but even trace with an average size of 1300-2000 bp.
   Tagmentation of cDNA and library prep with Nextera XT:

This step will tagment the DNA and add indexes to generate a sequencing library.
Preheat a thermocycler to 55° C.
For each sample, combine a minimum of 600 pg (ideally 1800 pg is) to a total volume of 5 μl.
Add 10 μl of Tagment DNA buffer (RD) and 5 μl of Amplicon Tagment Mix (ATM) bringing the total volume to 20μ;
Mix by pipetting up and down 5 times and spin to ensure liquid is at the bottom of the tube.
Incubate at 55° C. for 5 mins
Add 5 μl of Neutralization Tagment buffer (NT) and mix by pipetting up and down 5 times and spin down
Incubate at room temperature for 5 mins
Add into each PCR tube the following components in the order specified:

| Volume [μl] | Component |
|---|---|
| 15 | Nextera PCR MM (NPM), Kapa HiFi also works as a |
| | replacement |
| 8 | H₂O |
| 1 | 10 μM New-P5-SMART PCR oligo |
| 1 | 10 μM Nextera N70X oligo |

Run PCR program:

| Cycles | T [° C.] | Time | |
|---|---|---|---|
| | 95 | 30 | s |
| 12 | 95 | 10 | s |
| | 55 | 30 | s |
| | 72 | 30 | s |
| | 72 | 5 | min |

| | |
|---|---|
| 4 | ∞ |

Purification of cDNA and evaluate on Tapestation:
Vortex bottle of AMPPure beads to mix
Add 50 μl of H₂O to sample to make a final volume of 100 μl
Add 60 μl (0.6:1 beads to sample ratio, usually 250 μl) of AmpPure XP beads to each tube of sample.
Incubate 5 mins at room temperature
Place sample in magnet at high position
Transfer 150 μl of supernatant into a new tube
Add 20 μl of beads to the sample and mic up and down 5 times
Incubate for 5 mins at room temperature
Place in the magnet at high position
Remove supernatant and discard
Add 200 μl of 85% ethanol to wash the beads, wait 30 s
Remove the ethanol
Repeat ethanol wash twice
Centrifuge briefly and then return to magnet on low position
Remove residual ethanol and then wait for 2 mins
Elute in 20 μl of H₂O
Store at 4° C. for 72 hours or at −20° C. for long term storage

Run 2 μl of sample on a d1000 tapestation. Tagmented libraries should have a fairly smooth trace with an average size of 500-680 bp. You now have a library for sequencing which can be performed following illumine protocols.

### Example 2

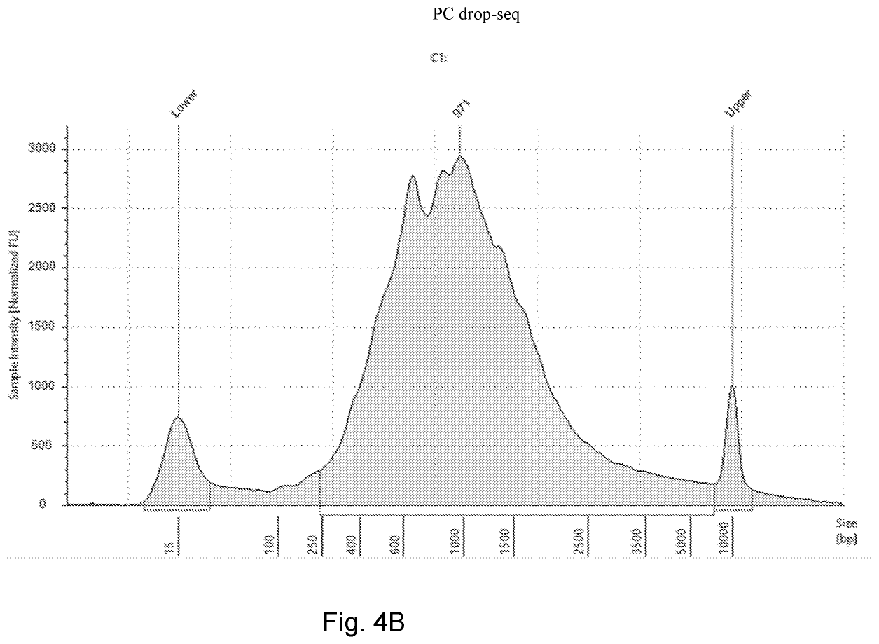

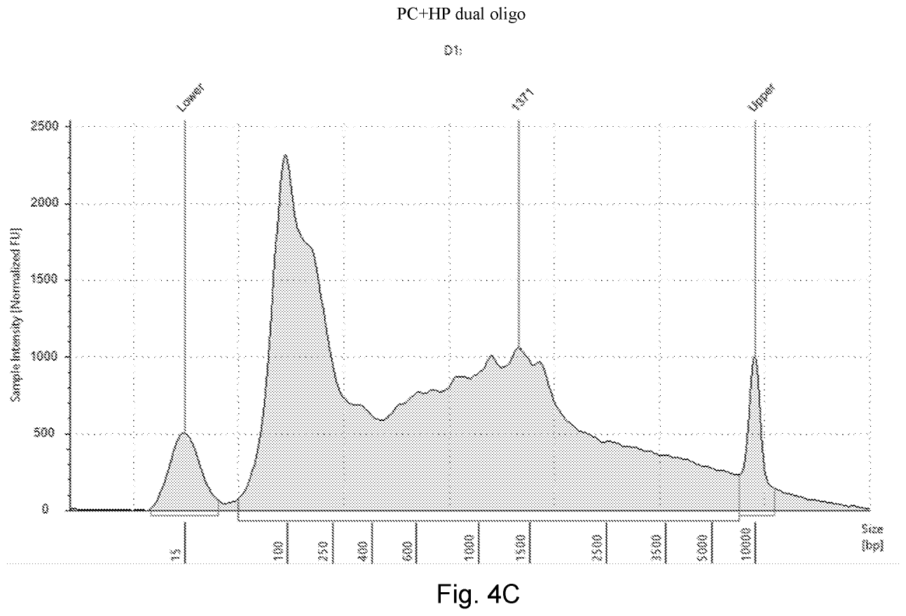

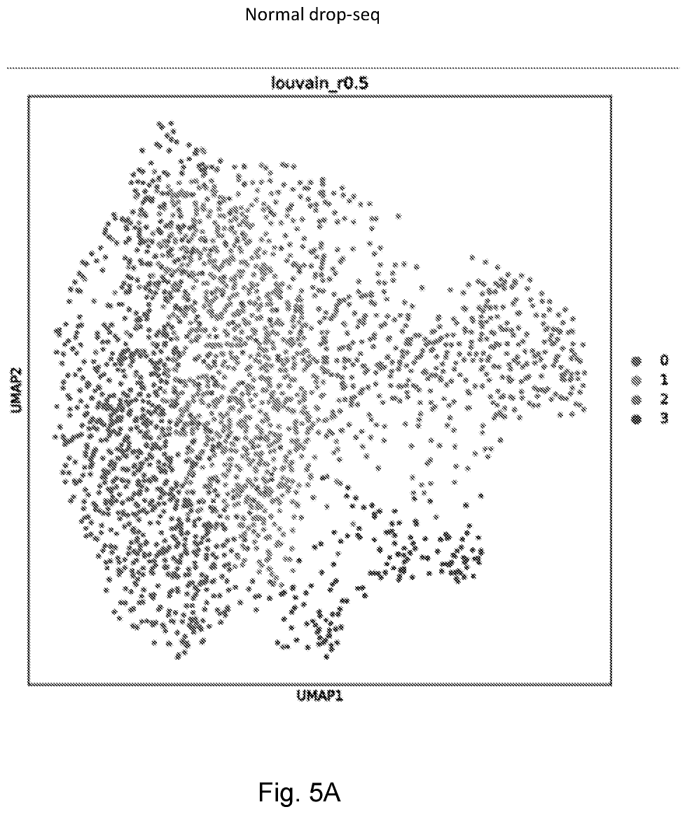

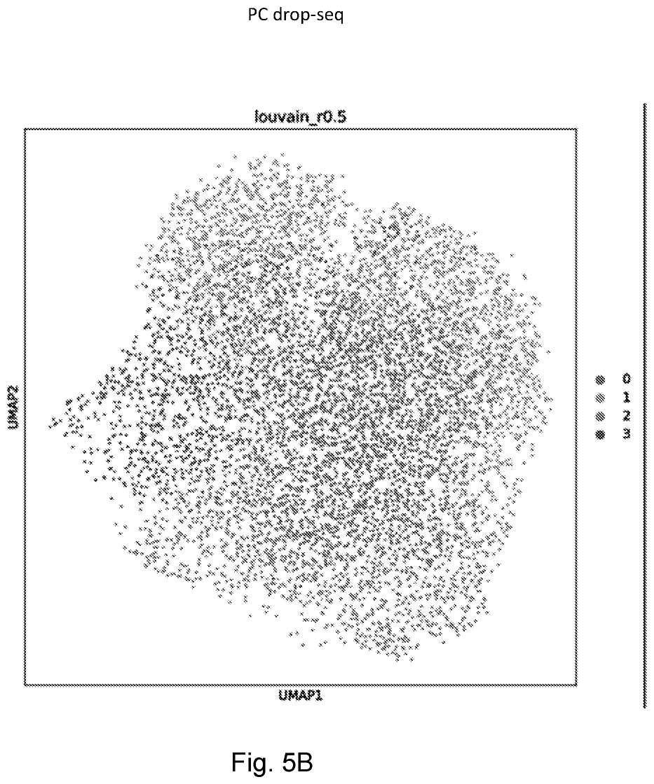

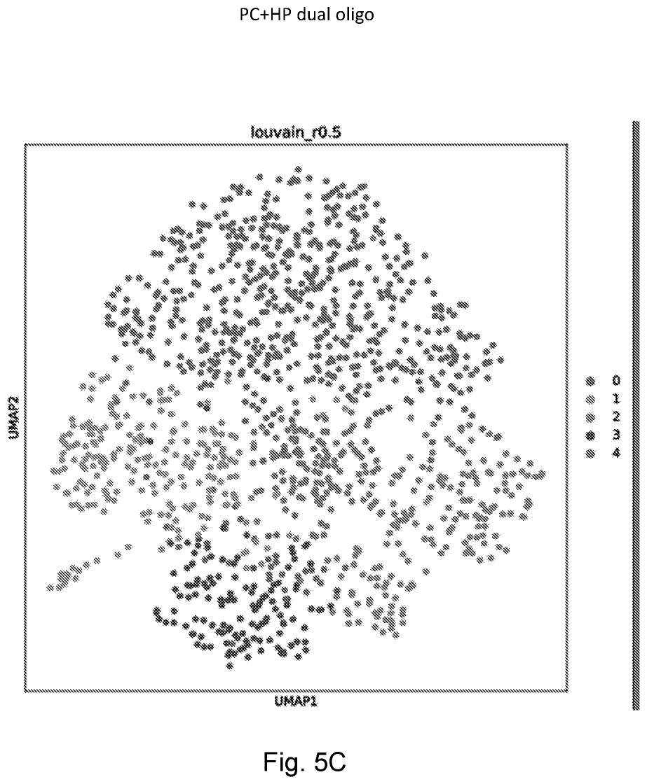

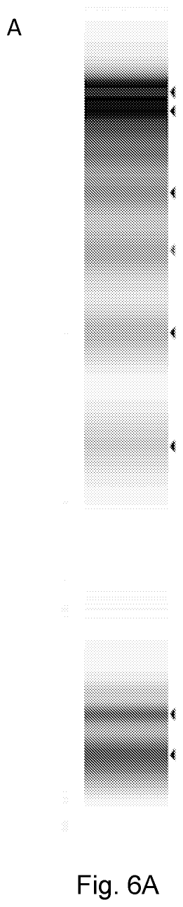

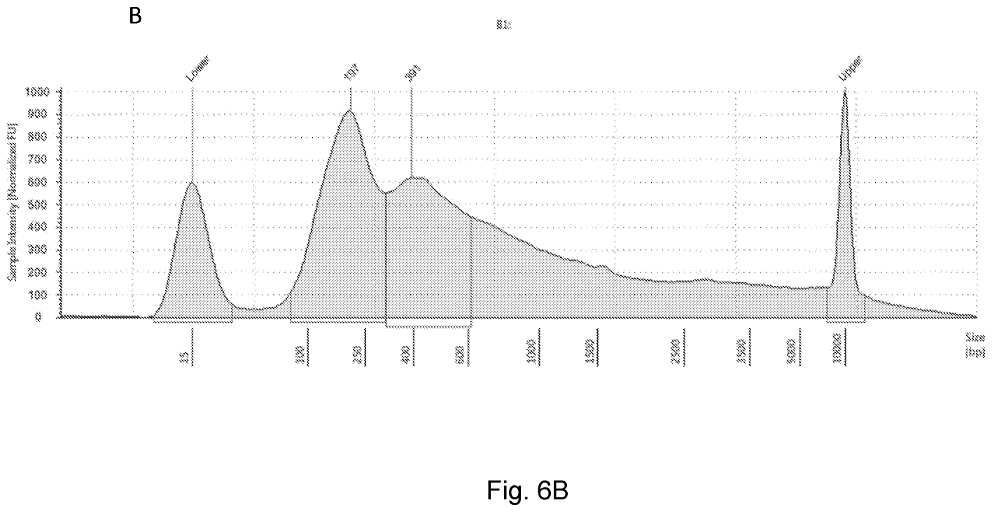

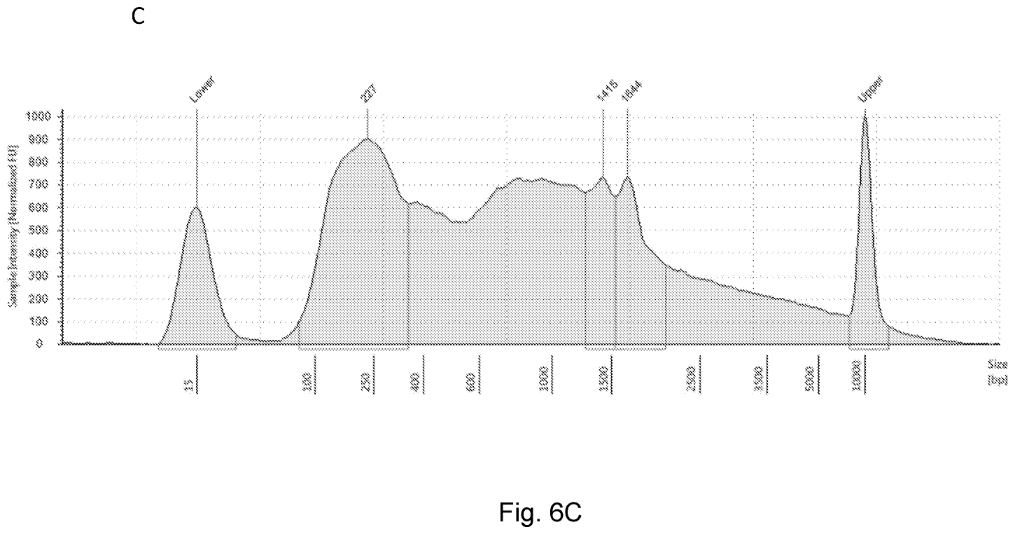

HEK293T cells were harvested and encapsulated as described in Example 1. Tapestation traces show the final library produced using three different protocols: A. Normal drop-seq using the bead sequence from the published EZ Macosko—2015 method for performing droplet based sequencing (FIG. 4A); B. PC drop-seq—This same sequence from EZ Macosko 2015, but with a photocleavable linker added at the 5′ end of the sequence; and C. PC+HP dual oligo-tapestation trace shows RNA library generated using the oligonucleotide sequence described in Example 1. The libraries were sequenced using Illumina Next seq 500 machine. UMAP plots show the number of cells measured using each of the three methods (FIG. 5, A-C). The results demonstrate that the bead oligonucleotides described in Example 1 are able to capture RNA that can be amplified to produce sequencing libraries of cellular RNA.

### Example 3—Exemplary Protocol for 3′ End mRNA Capture and 5′ End DNA Capture

The protocol is the same as set out in Example 1, except as follows:

Lysis buffer additionally includes 10 μL of KlenTaq and 10 μL T7 ligase. Cell buffer additionally includes 10 μL of blocking oligo.

### Primers:

| Name | Sequence |
|---|---|
| Template | 5′-AAGCAGTGGTATCAACGCAGAGTGAATrGrGrG |
| switch | |
| oligo | |
| SMART PCR | 5′-AAGCAGTGGTATCAACGCAGAGT |
| PRIMER | |
| New-P5- | 5′- |
| SMART | AATGATACGGCGACCACCGAGATCTACACGCCT |
| PCR | GTCCGCGGAAGCAGTGGTATCAACGCAGAGT* |
| hybrid | A*C |
| oligo | |
| Read1 | 5′-GCCTGTCCGCGGAAGCAGTGGTATCAACGCAG |
| Custom | AGTAC |
| primer | |
| Captured | 5′-TCGTCGGCAGCGTCAGATGTGTAT |
| DNA | |
| forward | |
| Captured | 5′-CGGATAACCTACGCTCGGAC |
| DNA | |
| reverse | |
| DNA final | 5′-CAAGCAGAAGACGGCATACGAGATTCGCCT |
| i7 | TAGTCTCGTGGGCTCGGAGATGTGTATAAGAG |
| | ACAGCGGATAACCTACGCTCGGAC |
| ATAC i5 | 5′-AATGATACGGCGACCAC |
| | CGAGATCTACACTCGTCGGCAGCGTCAGATGTG |
| MEDSB | 5′-P-GACGCTGCCGACG-InvA |
| Blocking | |
| oligo | |

Inv=Inverted base
Capture beads:

5′-TCGGACCGTTCGTCGGTGGTATCAACGCAGAGTACJJJJJJJJJJJJ
NNNNNNGTCCGAGCGTAGGTTATCCGTTTTTTTTTTTTTTTTTTTTTTTT
TTTTTTUCCGCG-HEG-CGCGGAAAAAA-HEG-PCLinker-T-Bead-
3′

Heat to remove Tn5 from the DNA:

This step is carried out in between incubation at 37 C to create the 3′ hydroxyl groups and the reverse transcription step, and removes the bound Tn5 from the DNA so the oligo can capture ATAC′d DNA.
Incubate the 1.5 ml tube at 72° C. for 5 mins
   PCR of cDNA using kapa HiFi:

Following addition of the SMART adapters following RT (using TSO) PCR amplification of the cDNA is performed using primers against the SMART sequence, as in the RNA-only protocol of Example 1. However, an additional initial PCR of 6 cycles is performed so that both ends of the oligo are amplified. Then the reactions are cleaned up and split between two PCR reactions, one to amplify the DNA captured end and the other to further amplify the RNA captured end.

Defrost master mix and SMART PCR primer

Place 24.6 μl of cDNA into a PCR tube and then add the following components:

| Volume (μl) | Component |
|---|---|
| 24.6 | cDNA |
| 0.4 | 100 μM Captured DNA F |
| 0.4 | 100 μM Captured DNA R |
| 0.4 | 100 μM SMART PCR primer |
| 25 | 2X Kapa HiFi MM |

Then proceed to PCR:

| Cycles | T [° C.] | Time | |
|---|---|---|---|
| | 95 | 3 | min |
| 4X | 98 | 20 | s |
| | 65 | 45 | s |
| | 72 | 3 | min |
| 1X | 98 | 20 | s |
| | 67 | 20 | s |
| | 72 | 3 | min |
| | 72 | 5 | mins |

| | |
|---|---|
| 4 | ∞ |

Purification of cDNA and evaluate on Tapestation:
Vortex bottle of AMPPure beads to mix
Add 0.6×(0.6:1 beads to sample ratio, usually 250 μl) of AmpPure XP beads to each tube of sample.
Perform the clean-up following manufacturers protocol
Elute in 20 μl of H₂O
   PCR amplify captured DNA:

This PCR will amplify the captured DNA end of the oligo and add the i7 and i5 adapters, which can then be directly sequenced on an illumina machine.
In a PCR add the following components:

| Volume (μl) | Component |
|---|---|
| 2.5 | 25 μM DNA final PCR primer |
| 2.5 | 25 μM ATAC i5 |
| 5 | DNA product from “PCR of cDNA using kapa HiFi” |
| 25 | 2X Kapa HiFi MM |

| Cycles | T [° C.] | Time | |
|---|---|---|---|
| | 95 | 3 | min |
| 4X | 98 | 20 | s |
| | 65 | 45 | s |
| | 72 | 3 | min |
| 5X | 98 | 20 | s |
| | 67 | 20 | s |
| | 72 | 3 | min |
| | 72 | 5 | mins |

| | |
|---|---|
| 4 | ∞ |

Then proceed to PCR:
   PCR amplify captured RNA end of the oligo:
Add the following components to a PCR tube:

| Volume (μl) | Component |
|---|---|
| 15 | DNA product from “PCR of cDNA using kapa HiFi” |
| 4.6 | H₂O |
| 0.4 | 100 μM SMART PCR primer |
| 25 | 2X Kapa HiFi MM |

Then perform PCA as follows:

| Cycles | T [° C.] | Time | |
|---|---|---|---|
| | 95 | 3 | min |
| 4X | 98 | 20 | s |
| | 65 | 45 | s |
| | 72 | 3 | min |
| 9X | 98 | 20 | s |
| | 67 | 20 | s |
| | 72 | 3 | min |
| | 72 | 5 | mins |

| | |
|---|---|
| 4 | ∞ |

Complete protocol as in Example 1.

### Example 4

HEKI293T cells were harvested and ATAC was performed then encapsulated as described in Example 3. Tapestation trace (FIG. 5) shows both a DNA library produced from the 5′ capture and an RNA library produced from the 3′ capture, the final library generated by amplifying the DNA hybridized at the 5′ end of the dual oligonucleotide. The results demonstrate that the bead oligonucleotides described in Example 3 are able to capture both RNA and DNA that can be amplified to produce sequencing libraries.

1. A micro-particle comprising a micro-bead and an array of polynucleotides, wherein each polynucleotide is attached to the micro-bead at the 3′ end, and wherein each polynucleotide comprises, in a 3′ to 5′ direction:
(a) a linker that is cleavable to provide a free 3′ hydroxyl group on the polynucleotide after cleavage;
(b) a 3′ end analyte capture region;
(c) a barcode sequence (BC), wherein the BC of each polynucleotide is the same; and a unique molecular identifier sequence (UMI), wherein the UMI of each polynucleotide is different; and wherein the UMI is 5′ or 3′ to the BC; and
(d) optionally a polymerase chain reaction (PCR) handle sequence.

2. The micro-particle of claim 1, wherein each polynucleotide comprises, in a 3′ to 5′ direction:
(a) a linker that is cleavable to provide a free 3′ hydroxyl group on the polynucleotide after cleavage;
(b) a 3′ end analyte capture region;
(c) optionally a first PCR handle sequence;
(d) a barcode sequence (BC), wherein the BC of each polynucleotide is the same; and a unique molecular identifier sequence (UMI), wherein the UMI of each polynucleotide is different; and wherein the UMI is 5′ or 3′ to the BC;
(e) optionally a second PCR handle sequence; and
(f) a 5′ end analyte capture region.

3. The micro-particle according to claim 1 or claim 2, wherein the linker comprises a photocleavable linker.

4. The micro-particle according to any one of claims 1 to 3, wherein the linker comprises a site for cleavage by one or more enzymes to provide the free 3′ hydroxyl group.

5. The micro-particle according to claim 4, wherein the linker comprises a site for cleavage by an exonuclease and a class I AP endonuclease to provide the free 3′ hydroxyl group.

6. An array of polynucleotides comprising, from 3′ to 5′:
(a) a 3′ hydroxyl group;
(b) a 3′ end analyte capture region;
(c) optionally a first polymerase chain reaction (PCR) handle sequence;
(d) a barcode sequence (BC), wherein the barcode sequence of each of the polynucleotides is the same; and a unique molecular identifier sequence (UMI), wherein the UMI of each polynucleotide is different; and wherein the UMI is 5′ or 3′ to the BC;
(e) optionally a second PCR handle sequence; and
(f) optionally a 5′ end analyte capture region.

7. The array of polynucleotides according to claim 6, wherein the array is bound to a micro-bead.

8. The micro-particle or array of polynucleotides of any one of the preceding claims, wherein the 3′ and/or 5′ end analyte capture region comprises
(a) a polythymidine sequence;
(b) an aptamer;
(c) a sequence of at least 10 nucleotides for hybridising to a target polynucleotide analyte;
(d) a biotinylated nucleotide sequence (e) an ATAC-med sequence.

9. The micro-particle or array of polynucleotides according to any one of the preceding claims, wherein the PCR handle sequence(s) are at least 15 nucleotides in length.

10. The micro-particle or array of polynucleotides according to any one of the preceding claims, wherein the BC is 10-14 nucleotides in length and/or the UMI is 6 to 10 nucleotides in length.

11. The array of polynucleotides according to any one of claims 6 to 10, wherein the 3′ end analyte capture region and/or the 5′ end capture region is hybridised to a polynucleotide analyte.

12. The array of polynucleotides according to any one of claims 6 to 11 wherein the 3′ end analyte capture region and/or the 5′ end capture region are bound to analyte, optionally wherein the analytes are
(i) mRNA;
(ii) DNA; and/or
(iii) protein.

13. The array of polynucleotides according to claim 12, wherein
(i) the 3′ end analyte capture regions are polythymidine and the analytes comprise mRNA bound to the polythymidines;
(ii) the 3′ end and/or 5′ end capture regions are aptamers and the analytes comprise protein bound to the aptamers;
(iii) the 3′ end and/or 5′ end capture regions are polynucleotide sequences and the analytes comprise polynucleotides hybridised to the polynucleotide capture regions;
(iv) the 3′ end and/or 5′ end capture region are biotinylated regions and the analytes comprise streptavidin or avidin bound to the biotinylated regions.

14. The array of polynucleotides according to any one of claims 11 to 13, wherein the analytes bound to the polynucleotides of the array are from a single cell or cell nuclei.

15. A plurality of micro-particles according to any one of claims 1 to 5 and 8 to 10, or a plurality of micro-particles each comprising a micro-bead bound to an array of polynucleotides according to any one of claims 6 to 14, wherein the array of each micro-particle has a different BC from the array of essentially each other micro-particle.

16. A fluidic compartment, optionally a microfluidic compartment, comprising a single micro-particle or a single array of polynucleotides according to any one of claims 1 to 15 and optionally a single cell, a single cell nucleus, a single vesicle, a single cell lysate, a single cell nucleus lysate, or a single vesicle lysate.

17. A method of synthesising an array of polynucleotides on the surface of a micro-bead, wherein the polynucleotides are synthesized in a 3′ to 5′ direction from the bead to the polynucleotide free ends and wherein each polynucleotide comprises, from 3′ to 5′:
(a) a linker that is cleavable to provide a free 3′ hydroxyl group on the polynucleotide after cleavage from the micro-particle;
(b) a 3′ end analyte capture region;
(c) optionally a first polymerase chain reaction (PCR) handle sequence;
(d) a barcode sequence (BC), wherein the barcode sequence of each of the polynucleotides is the same; and a unique molecular identifier sequence (UMI), wherein the UMI of each polynucleotide is different; and wherein the UMI is 5′ or 3′ to the BC;
(e) optionally a second PCR handle sequence; and
(f) optionally a 5′ end analyte capture region.

18. A method for generating one or more libraries from one or more groups of analytes from the same sample, wherein the method comprises
(i) contacting the sample with an array of polynucleotides according to any one of claims 6 to 14;
(ii) allowing analytes to bind to the 3′ end and/or 5′ end analyte capture regions of the polynucleotides; and
(iii) generating one or more libraries from the analytes bound to the 3′ end and/or 5′ end analyte capture regions, optionally wherein the method comprises generating a first library from the analytes bound to the 3′ end analyte capture regions, and generating a second library from the analytes bound to the 3′ end analyte capture regions.

19. The method of claim 18, wherein the 3′ end capture regions bind to RNA in the sample and the method comprises
(iv) reverse transcription using the bound RNA as template to provide an RNA/cDNA hybrid;
(v) template switch to extend the end of the RNA/cDNA hybrid to include a template switch PCR handle sequence; and
(vi) PCR amplification using primers that hybridize to (A) the template switch PCR handle sequence and (B) the PCR handle sequence 5′ to the BC and UMI sequence.

20. The method according to claim 18 or 19, wherein
(a) the 5′ end capture region binds to DNA in the sample, and wherein the method comprises PCR amplification using a pair of PCR primers that hybridize to (A) a PCR handle on the DNA bound to the 5′ end capture region, and (B) the complement of the PCR handle sequence 3′ to the UMI and BC; and/or
(b) the 3′ end capture region binds to DNA in the sample, and wherein the method comprises PCR amplification using a pair of PCR primers that hybridize to (A) a PCR handle on the DNA bound to the 3′ end capture region, and (B) the PCR handle sequence 5′ to the UMI and BC.

21. The method according to any one of claims 18 to 20, wherein the one or more groups of analytes are from a single cell, single cell nucleus or single vesicle.

22. The method of claim 21, wherein the method comprises:
(i) isolating the single cell or single cell nucleus or single vesicle with a single micro-particle according to any one of claims 1 to 5 and 8 to 10 in a fluidic compartment, optionally a microfluidic compartment;
(ii) lysing the cell, cell nucleus or vesicle; and
(iii) cleaving the linker to provide the array of polynucleotides.

23. The method according to any one of claims 18 to 20, wherein the analytes are from a sample comprising a plurality of cells or cell nuclei or vesicles and the method comprises:
(i) isolating a single cell or single cell nuclei or single vesicle of the sample and a single micro-particle according to any one of claims 1 to 5 and 8 to 11 in each of a plurality of separate fluidic compartments, wherein the polynucleotide array of essentially each micro-particle has a different barcode sequence;
(ii) lysing the isolated cells, cell nuclei or vesicles;
(iii) cleaving the linker of the polynucleotides to provide an array of polynucleotides with free 3′ hydroxyl groups in each fluidic compartment.

24. The multiplex method according to any one of claims 18 to 23, wherein the method further comprises:
(I) one or more further rounds of PCR amplification, wherein each round uses a pair of primers that hybridize to
(A) the template switch handle sequence(s) and the PCR handle sequence 5′ to the UMI and BC; or
(B) a PCR handle on a DNA analyte and the complement of the first PCT template handle; and/or
(II) sequencing one or more of the libraries.


SEQUENCE LISTING (20 sequences)

SEQ ID NO: 1 - DNA, 17 bp, Artificial Sequence
  Linker
  misc_feature (6)..(7): HEG
  misc_feature (17)..(17): 3′ 17-O-(4,4′-Dimethoxytrityl)- hexaethyleneglycol, 1-[(2-cyanoethyl)- (N,N-diisopropyl)]-phosphoramidite (HEG)
uccgcgcgcggaaaaaa

SEQ ID NO: 2 - DNA, 16 bp, Artificial Sequence
  Linker
  misc_feature (8)..(9): HEG
gaagagctagctcttc

SEQ ID NO: 3 - DNA, 14 bp, Artificial Sequence
  Linker
  misc_feature (7)..(8): HEG
tgtacatatgtaca

SEQ ID NO: 4 - DNA, 20 bp, Artificial Sequence
  PCR Handle
gtggtatcaacgcagagtac

SEQ ID NO: 5 - DNA, 20 bp, Artificial Sequence
  PCR Handle
gtccgagcgtaggttatccg

SEQ ID NO: 6 - DNA, 30 bp, Artificial Sequence
  Template Switch Oligo
  misc_feature (28)..(30): ribonucleic acid Guanine
aagcagtggtatcaacgcagagtgaatggg

SEQ ID NO: 7 - DNA, 23 bp, Artificial Sequence
  SMART PCR PRIMER
aagcagtggtatcaacgcagagt

SEQ ID NO: 8 - DNA, 66 bp, Artificial Sequence
  New-P5-SMART PCR hybrid oligo
  misc_feature (65)..(66): Phosphorothioated DNA nucleotides
aatgatacggcgaccaccgagatctacacgcctgtccgcggaagcagtggtatcaacgcagagtac

SEQ ID NO: 9 - DNA, 37 bp, Artificial Sequence
  Read1 Custom primer
gcctgtccgcggaagcagtggtatcaacgcagagtac

SEQ ID NO: 10 - DNA, 120 bp, Artificial Sequence
  capture bead sequence
  misc_feature (36)..(53): 12 nucleotide barcode sequence - 6 nucleotide UMI sequence
  misc_feature (109)..(110): HEG
  misc_feature (120)..(120): 5′ HEG, PCLinker, T bead
tcggaccgttcgtcggtggtatcaacgcagagtacnnnnnnnnnnnnnnnnnngtccgagcgtaggttatccgttttttttttttttttttttttttttttttuccgcgcgcggaaaaaa

SEQ ID NO: 11 - DNA, 24 bp, Artificial Sequence
  Captured DNA forward
tcgtcggcagcgtcagatgtgtat

SEQ ID NO: 12 - DNA, 20 bp, Artificial Sequence
  Captured DNA reverse
cggataacctacgctcggac

SEQ ID NO: 13 - DNA, 86 bp, Artificial Sequence
  DNA final i7
caagcagaagacggcatacgagattcgccttagtctcgtgggctcggagatgtgtataagagacagcggataacctacgctcggac

SEQ ID NO: 14 - DNA, 50 bp, Artificial Sequence
  ATAC i5
aatgatacggcgaccaccgagatctacactcgtcggcagcgtcagatgtg

SEQ ID NO: 15 - DNA, 14 bp, Artificial Sequence
  MEDSB Blocking oligo
  misc_feature (14)..(14): inverted base
  misc_feature (14)..(14): 5′ phosphate
gacgctgccgacga

SEQ ID NO: 16 - DNA, 17 bp, Artificial Sequence
  polyA
aaaaaaaaaaaaaaaaa

SEQ ID NO: 17 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 419 aa, SARS-Cov-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 19 - protein, 75 aa, SARS-Cov-2
MYSFVSEETGTLIVNSVLLFLAFVVFLLVTLAILTALRLCAYCCNIVNVSLVKPSFYVYSRVKNLNSSRVPDLLV

SEQ ID NO: 20 - protein, 222 aa, SARS-Cov-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ